(19) United States
(12) Patent Application Publication
(10) Pub. No.: US 2020/0373014 A1
(43) Pub. Date: Nov. 26, 2020

(54) DETERMINATION OF LIKELIHOOD OF SUFFERING RENAL DISEASE

(71) Applicants: Yuan-Hau LIN, (US); Hsien-Shou KUO, (US); Chiaolong HSIAO, (US)

(72) Inventors: Yuan-Hau LIN, Taipei City (TW); Hsien-Shou KUO, Taipei City (TW); Chiaolong HSIAO, Taipei (TW)

(21) Appl. No.: 16/881,588

(22) Filed: May 22, 2020

Related U.S. Application Data
(60) Provisional application No. 62/851,808, filed on May 23, 2019.

(51) Int. Cl. G16H 50/20; G16C 20/00; G16H 50/30; G16H 10/60; G16H 10/40; G16H 50/70

(52) U.S. Cl. CPC G16H 50/20; G16C 20/00; G16H 50/70; G16H 10/60; G16H 10/40; G16H 50/30

(57) ABSTRACT

The disclosure relates to determination of likelihood of suffering a renal disease. Particularly, the disclosure relates to using data derived from isotopic ratios of hydrogen and oxygen of water in biological samples for determination of likelihood of suffering a renal disease.

### RELATED APPLICATIONS

This application claims the benefit and priority to U.S. Provisional Application No. 62/851,808, filed on May 23, 2019, entitled, “DETERMINATION OF LIKELIHOOD OF SUFFERING RENAL DISEASE”, the contents of which is incorporated by reference herewith in its entirety.

### FIELD OF THE INVENTION

The instant disclosure relates to methods for determining the likelihood of suffering a renal disease. Particularly, the disclosure relates to using data derived from isotopic ratios of hydrogen and oxygen of water in biological samples for determination of likelihood of suffering a renal disease.

### BACKGROUND OF THE INVENTION

The human body contains 70% water and water is usually considered as a solvent.

Nevertheless, water molecules also participate bio-chemical reactions occurred in the human body and thus water can also act as a “solvate.”

The kidney is either one or a pair of organs in the dorsal region of the vertebrate abdominal cavity, functioning to maintain proper water and electrolyte balance, regulate acid-base concentration, and filter the blood of metabolic wastes, which are excreted as urine.

Thus, the consequence of a kidney disorder can constitute an overall imbalance in the organism as a whole. Many organs such as the bladder, intestine, heart, lungs and prostate depend on the ability of the kidney to filter out the undesirable debris of the body and maintain overall homeostasis.

Kidneys are essentially blood-cleansing organs. The renal artery from the heart brings blood into the kidneys to be cleaned by a network of millions of glomerulus containing nephrons. The nephrons filter out toxins, excess nutrients and body fluids. The remaining cleaned and filtered blood then passes through the renal veins back into circulation. The filtered out material travels down a tubule that adjusts the level of salts, water and wastes that are excreted in the urine. The renal pelvis collects the urine. From the pelvis, urine travels down the ureter into the urinary bladder. The urine is expelled from the bladder and out of the body through the urethra.

Types of kidney disease include diabetes, high blood pressure, glomerulonephritis and cysts. Diabetes affects the body's ability to regulate glucose. Excess glucose in the blood can damage the nephrons in the kidneys reducing the blood vessels' ability to filter toxins. High blood pressure can also damage the nephrons. Glomerulonephritis generally relates to a class of other diseases not related to kidney infection.

If both kidneys stop functioning due to disease, patients experience end-stage renal disease (ESRD), or total kidney failure. Kidney failure means that the body can no longer filter and remove certain toxins and cannot properly regulate blood pressure and critical nutrients. Unless kidney failures are treated, patients can die within days due to the build-up of toxins and fluid in their blood.

Diabetic kidney disease, also known as diabetic nephropathy, is an important cause of excess morbidity and premature mortality in individuals with type 1 diabetes mellitus (T1DM). Approximately 25% to 40% of patients with T1DM ultimately develop diabetic nephropathy. The most serious long-term effect of diabetic nephropathy is kidney failure leading to end stage renal disease (ESRD), a condition in which there is a permanent and almost complete loss of kidney function, with the kidneys functioning at less than 10% of baseline function. Other causes of ESRD include high blood pressure, glomerulonephritis, polycystic kidneys, interstitial disease, obstructive uropathy, systemic lupus erythematosus, and multiple myeloma.

Water (H₂O) is the base of life, yet the most important and the most abundant substance in human body, making up about seventy percent of body mass. Despite the awareness of the importance of water, an often-overlooked facet is the uniqueness of the water isotopes. Natural water contains trace amount of heavy isotope hydrogen and oxygen atoms, and among which the ²H and ¹⁸O are the major ones. The ratio of ¹H to ²H is about 6240 to 1, or about 155 ppm in the V-SMOW (Vienna Standard Mean Ocean Water) water standard, and the ratio of ¹⁶O to ¹⁸O is about 499 to 1, or about 2005 ppm. The unique disposition of isotope water and with the rapid advancement of the mass spectrometry technology, the stable isotopic ratios of hydrogen (δ²H) and oxygen (δ¹⁸O ) in various biological tissues have been used as “atomic fossils or tracers” in paleodietary, meteorology, anthropology, ecology, and modern food-chained network.

The stable nonradioactive isotope water, ²H₂O, could play important roles in human physiology and pathophysiology. Several studies using model animals and cell cultures have shown that the augment or depletion of ²H₂O in the dietary water has prominent effects on pathophysiology and physiology. For example, it was previously shown that the ²H₂O can promote the formation of microtubules by stimulating the polymerization of tubulin subunits and result in cell death. In addition, the increase of ²H₂O content can prevent hypertension in the spontaneous hypertension rat. On the other hand, the depletion of deuterium in the water of culture medium reduces the growth rates of different animal cell lines. It was also showed that the signatures of hydrogen (δ²H) and oxygen (δ¹⁸O) isotope ratios in the body water of an untreated streptozotocin-induced diabetes mellitus are distinct from those of the normal mice.

However, none of the prior art references discloses, teaches or suggests a robust manner of using data derived from water isotopes for determination of likelihood of suffering renal diseases.

### SUMMARY OF THE INVENTION

The present disclosure relates to a method for diagnosing renal disease, including: receiving first data, second data and third data associated with water molecules;
selecting a first parameter associated with the first data and a second parameter associated with the second data;
establishing a relation between the first data and the second data based on the first parameter and the second parameter;
obtaining a threshold based on the relation between the first data and the second data; and comparing the third data to the threshold to obtain a first probability of suffering a renal disease in a subject suspected of the renal disease,
wherein the first, second and third data are derived from isotopic information of hydrogen and oxygen of water molecules of a biological sample of target human.

The present disclosure also relates to an apparatus, including:
a control unit; and
a memory including computer program code;
wherein the memory and the computer program code are configured to, with the control unit, cause the apparatus to perform the method as noted above, including receiving and processing the first, second and third data derived from isotopic information of hydrogen and oxygen of water molecules of a biological sample of target human.

### BRIEF DESCRIPTION OF THE DRAWINGS

FIG. 1 illustrates an exemplified apparatus for conducting a method for diagnosing renal disease in accordance with some embodiments of the present disclosure.

FIGS. 2-8 show the diagrams obtained by using different models for the determination of likelihood of suffering renal disease.

### DETAILED DESCRIPTION OF THE INVENTION

The subject application surprisingly found that using statistical methods to verify the significant difference of data derived from the isotopic information of hydrogen and oxygen of water molecules of a biological sample of target human may provide helpful information about the likelihood of suffering from renal disease.

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety. In case of conflict, the definitions in the present specification will be referred to. In addition, the materials, methods, and examples are illustrative only and not intended to be limiting.

The term “detecting” or “diagnosing” refers to identifying a patient with renal disease and/or a patient who is at risk for having a renal disease.

The term “assay” means an analysis done on a sample to determine the presence of a substance and/or the amount of that substance in the sample.

The term “decreased amount” means an amount that is lower as compared to a predetermined level.

The term “renal disease” means the disorder, impairment, abnormal condition or dysfunction of an individual's renal function.

The term “sample” means a body fluid or tissue fluid sample obtained from a mammalian subject. As a non-limiting example, a body fluid or tissue fluid sample for use in the present invention can be urine, blood, serum, plasma, saliva, lymph, cerebrospinal fluid, cystic fluid, ascites, stool, bile, tissue fluid, and any other isolatable body fluid.

All publications and patent documents cited in this application are incorporated by reference in their entirety for all purposes to the same extent as if each individual publication or patent document were so denoted. By their citation of various references in this document, Applicants do not admit any particular reference is “prior art” to their invention.

It could be understood or speculated that the δ²H and δ¹⁸O isotope ratios of water molecules in humans who are healthy might fall within a certain range. However, the range may be broad and thus taking the values as the mere indicator for determination of likelihood of suffering renal disease may not be precise enough. In addition, how to determine a threshold value would be a crucial problem.

On the other hand, it could be postulated that each value may be deemed as being randomly distributed in said range if a sufficient amount of data is collected and analyzed. To this end, the subject application developed several manners to express the phenomena.

In one embodiment, by conducting a conceptual experiment wherein the values derived from δ⁸H and δ¹⁸O isotope ratios of water molecules in humans are considered as an ensemble defined in statistical thermodynamics, then it can assume each value as a microstate and calculate the entropy of each microstate. In this experiment, it is assumed that the biological reactions which involve water in a healthy human body as the “normal state” and in a human suffering from renal disease(s) as the “abnormal state.” Given this, it would be obvious that the two states would be significantly different from each other and the difference may cause different (total) entropy and distribution of entropy.

In statistic thermodynamics, entropy can be expressed by the following formula:

S=−ΩlnΩ,

wherein Ω is the number or probability of (micro)states.

In the case of an ensemble of δ²H and δ¹⁸O isotope ratios of water molecules in humans, the absolute concentration of hydrogen and oxygen isotopes can be considered as Ω. The values of δ²H and δ^{˜}O can be measured and calculation can be performed based on these values to obtain the absolute concentration. The means for measuring the value of the delta O-18 or delta H-2 can be selected from any suitable means for proving/quantifying the delta O-18 or delta H-2, e.g., isotope ratio mass spectrometer. The entropy of each (micro)state can be obtained after obtaining the absolute concentration.

In one exemplary embodiment based on this connection, two ensembles can be chosen wherein one consists of human with no renal diseases (Group I) and the other consists of human subjected to hemodialysis (Group II). Then, a statistical method can be used to reveal the difference between the microstates in the two ensembles. It is found that the entropy of each microstate (e.g., each subject) of Group I exhibits randomly distribution but the entropy of each microstate of Group II falls within a much narrower range, e.g., average value ±1.5σ. On the other hand, when the microstates of Group I whose entropy falls within the range constructed by the entropy of each microstate of Group II, most subjects have family history of being subjected to hemodialysis or have diabetes.

Hence, the subject application provides a method for diagnosing renal disease, including:
receiving first data, second data and third data associated with water molecules;
selecting a first parameter associated with the first data and a second parameter associated with the second data;
establishing a relation between the first data and the second data based on the first parameter and the second parameter;
obtaining a threshold based on the relation between the first data and the second data; and
comparing the third data to the threshold to determine a first probability of suffering a renal disease in a subject suspected of the renal disease; wherein:
the first data include values of δ¹⁸O and δ²H of water molecules in blood samples of subjects who are not suffered from renal disease;
the second data include values of δ¹⁸O and δ²H of water molecules in blood samples of subjects who are diagnosed of suffering renal disease and are subjected to treatment of hemodialysis;
the third data include values of δ¹⁸O and δ²H of water molecules in a blood sample of the subject suspected of the renal disease; and
the first, the second and the third parameters are derived from application of logarithm, addition, deduction, multiplication, division or any combinations thereof on the first, the second and the third data.

In one embodiment, the method further includes obtaining the first, the second and the third data from medical record, measurement of the blood samples, or both.

In one embodiment, the method further includes obtaining forms of entropy of the first, second and third parameters.

In one embodiment, the method further includes obtaining the threshold based on one or more statistic models. In one embodiment, the statistic models include Student's t-test, F-test, Z-test, Analysis of variance (ANOVA) or any combinations thereof.

In one embodiment, the method further includes establishing the relation between the first data and the second data, and providing a two-dimensional plot with the first and second parameters as the axes. In one embodiment, the threshold is expressed with standard deviation (σ) or variance (σ²). In one embodiment, the threshold is defined by the range within ±2.0 σ, preferably ±1.5 σ, more preferably ±1.0 σ from the average value of the parameter.

In one embodiment, the blood sample includes blood plasma, erythrocytes or both.

In one embodiment, the subjects associated with the second data have a family medical history of being subjected to treatment of hemodialysis, diabetes or both.

In one related aspect, the exemplary methods described above are performed on a suitably configured apparatus, wherein the apparatus includes: a control unit; and a memory including computer program code; wherein the memory and the computer program code are configured to operate with the control unit, causing the apparatus to perform the method as noted above, including receiving and processing the first, second and third data sets derived from isotopic information of hydrogen and oxygen of water molecules of a biological sample of target human subject.

The subject application also provides an apparatus, including:
a control unit; and
a memory including computer program code;
wherein the memory and the computer program code are configured to, with the control unit, cause the apparatus to perform:
receiving first data, second data and third data associated with water molecules;
selecting parameters associated with the first data and the second data;
establishing a relation between the first data and the second data based on the first parameter and the second parameter;
obtaining a threshold based on the relation between the first data and the second data; and
comparing a third parameter associated with the third data to the threshold to determine a first probability of suffering a renal disease in a subject suspected of the renal disease; wherein:
the first data include values of δ¹⁸O and δ²H of water molecules in blood samples of subjects who are not suffered from renal disease;
the second data include values of δ¹⁸O and δ²H of water molecules in blood samples of subjects who are diagnosed of suffering renal disease and are subjected to treatment of hemodialysis;
the third data include values of δ¹⁸O and δ²H of water molecules in a blood sample of the subject suspected of the renal disease; and
the parameters are derived from application of logarithm, addition, deduction, multiplication, division or any combinations thereof on the first, the second and the third data.

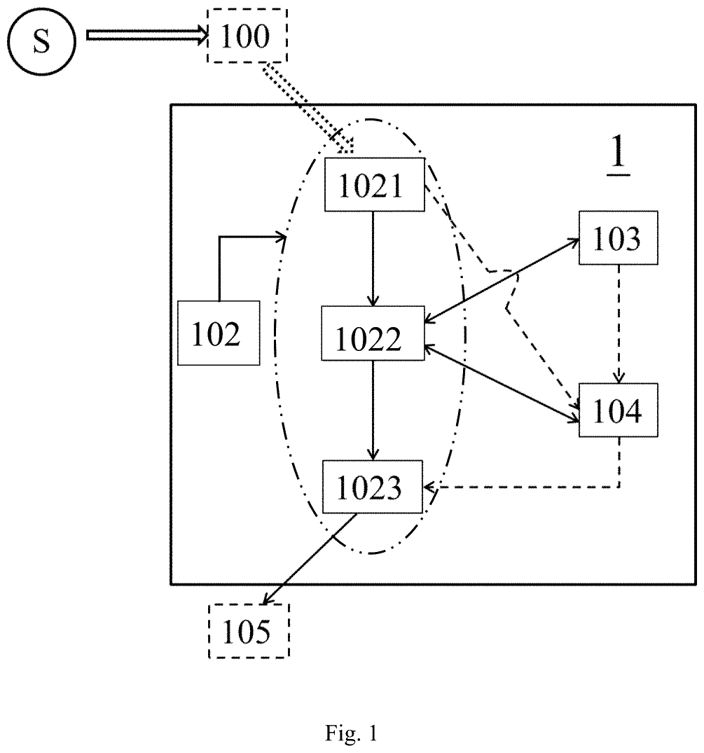

In one embodiment, the apparatus 1 as disclosed herein may be illustrated by FIG. 1, including:
a control unit 102, wherein the control unit comprises at least one receiving unit 1021, at least one processing unit 1022 and at least one output unit 1023; and
a memory 103 including computer program code 103a (not shown);
wherein the memory 103 and the computer program code 103a are configured to, with the control unit 102, cause the apparatus to perform:
receiving first data associated with water molecules from the receiving unit 1021;
receiving second data associated with water molecules from the receiving unit 1021;
receiving third data associated with water molecules from the receiving unit 1021;
determining parameters associated with the first data and the second data by the processing unit 1022;
determining two of the parameters to establish a relation between the first data and the second data by the processing unit 1022, wherein one parameter is associated with the first data and the other parameter is associated with the second data;
determining a threshold based on the relation between the first data and the second data by the processing unit 1022;
transmitting the data and/or parameters to the memory 103 forth and back for storing; comparing the third data to the threshold by a comparison unit 104 to determine a first probability of suffering a renal disease in a subject suspected of the renal disease;
transmitting result from the comparison unit 104 to the processing unit 1022; and
transmitting the result from the processing unit 1022 to an output unit 1023;
wherein:
the first data include values of δ¹⁸O and δ²H of water molecules in blood samples of subjects who are not suffered from renal disease;
the second data include values of 6¹⁸0 and 6²H of water molecules in blood samples of subjects who are diagnosed of suffering renal disease and are subjected to treatment of hemodialysis;
the third data include values of δ¹⁸O and δ²H of water molecules in a blood sample of the subject suspected of the renal disease; and
the parameters are derived from application of logarithm, addition, deduction, multiplication, division or any combinations thereof on at least one of the first and the second data.

In one embodiment, the apparatus disclosed herein receives the data from a data collecting unit/device 100, wherein the data is collected from a subject(s) S.

In one embodiment, the result of comparison obtained in the comparison unit 104 is transmitted back to the processing unit 1022; in another embodiment, the result of comparison obtained in the comparison unit 104 is transmitted to the output unit 1023.

In one embodiment, a display unit/device 105 receives the information from the apparatus 1, in particular from the output unit 1023, and displays the outcome of the diagnosis.

In one embodiment, the third data is directly transmitted to the comparison unit 104.

In one embodiment, the data/parameter(s) stored in the memory 103 is transmitted to the comparison unit 104.

In one embodiment, the parameters are expressed in the form of entropy.

In one embodiment, the threshold is obtained based on one or more statistic models. In one embodiment, the statistic models include Student's t-test, F-test, Z-test, Analysis of variance (ANOVA) or any combinations thereof.

In one embodiment, the relation is established by providing a two-dimensional plot with the first and the second parameters as the axes. In one embodiment, the threshold is expressed with standard deviation (σ) or variance (σ²). In one embodiment, the threshold is defined by the range within ±2.0 σ, preferably ±1.5 σ, more preferably ±1.0 σ from the average value of the parameter.

In one embodiment, the blood sample is blood plasma, erythrocytes or both.

In one embodiment, the subjects associated with the second data have a family medical history of being subjected to treatment of hemodialysis, diabetes or both.

### EXAMPLE

While the invention has been described with reference to specific embodiments, it will be understood by those skilled in the art that various changes may be made and equivalents may be substituted for elements thereof to adapt to particular situations without departing from the scope of the invention. The following experimental examples are provided in order to demonstrate and further illustrate various aspects of certain embodiments of the present invention and are not to be construed as limiting the scope thereof In the experimental disclosure which follows, the following materials and methods are used:

Participants.

Human subjects.

Water samples. 3 ml of human blood plasma sample is stored in a 15 ml falcon tube. The tube is then placed into a pre-dried vacuumed round bottle flask with 15 g of CaCl₂ granule (Sigma-Aldrich). The round bottle flask is then capped and sealed carefully to make sure no water in air gets into the flask. The flask is incubated at room temperature for CaCl₂ to absorb water from the human blood plasma sample for seven days. The water sample (about 2 ml) is obtained from the hydrated CaCl₂ by vacuum distillation (Buchi Glass Oven B-585, Kugelrohr).

Determination of hydrogen (δ²H) and oxygen (δ¹⁸O) in human blood plasma. The assessment of hydrogen (67 ²H) and oxygen (δ¹⁸O) in the water samples was conducted as the following. The stable oxygen isotopic compositions were analyzed by the well-known CO₂-H₂O equilibration method (Epstein S, Mayeda T (1953) Variation of O18 content of waters from natural sources. Geochimica et Cosmochimica Acta 4: 213-224; Brenninkmeijer CAM, Morrison PD (1987) An automated system for isotopic equilibration of CO2 and H2O for 18O analysis of water. Chemical Geology: Isotope Geoscience section 66: 21-26). The equilibrated CO₂ gas was measured by a VG SIRA 10 isotope ratio mass spectrometer. The hydrogen isotopic compositions were determined on a VG MM602D isotope ratio mass spectrometer after reduction of water to H₂ using Zinc shots made by Biogeochemical Laboratory of Indiana University (Coleman M L, Shepherd T J, Durham J J, Rouse J E, Moore G R (1982) Reduction of water with zinc for hydrogen isotope analysis. Analytical Chemistry 54: 993-995). All isotopic ratio results are reported as the δ-notation (‰) relative to the international V-SMOW (Vienna Standard Mean Ocean Water) standard and normalized on the scale that the δ ¹⁸O and δ²H of SLAP (Standard Light Antarctic Precipitation) are -55.5 ‰ and −428 ‰, respectively (Gonfiantini R (1978) Standards for stable isotope measurements in natural compounds. Nature 271: 534-536). The analytical precisions expressed as 1σ for the laboratory standards are better than 1.3 ‰ for δ²H and 0.08 ‰ for δ¹⁸O, respectively. The average differences of duplicate analyses of water samples are ±1.5‰ for δ²H and ±0.11 ‰ for δ¹⁸O, respectively.

Date Analysis. The ANOVA and the Student's t-test were performed by using the STASTISTICA 8.0 (StartSoft. Inc., Tulsa, Okla.). The k-means clustering was performed in the MATLAB R2011a (MathWorks. Inc., Natick, Mass.) that data are partitioned into a preset of four clusters. Euclidean distance is measured to compute the centroid of cluster, a mean of the data points within that cluster. A total number of 10,000 repeated clustering processes were performed, as a new set of initial cluster centroid position was given in each round. This procedure returns the best solution of a four clusters that each cluster is with the lowest value of sum of point-to-centroid distances.

### Example 1

The following data were collected for the diagnosis of likelihood of suffering a renal disease:

A first data comprising values of δ¹⁸O0 and 6²H of water molecules in blood samples of subjects who are not suffered from renal disease (Group I), a second data comprising values of δ¹⁸O and δ²H of water molecules in blood samples of subjects who are diagnosed of suffering renal disease and are subjected to treatment of hemodialysis (Group II), and a third data comprising values of 6¹⁸0 and 6²H of water molecules in a blood sample of the subject suspected of the renal disease (Target).

Then, calculating the following parameters from the values of δ¹⁸O and δ²H of each group:

R²H=²H/¹H; R¹⁸O=¹⁸O/¹⁶O;

S=−Ωln(Ω).

R²H and R¹⁸O are applied as Ω in the formula noted above to obtain S(R²H) and S(R¹⁸H), respectively.

rₚ=S (Rₚ²H)/ S (Rₚ¹⁸O); rₑ=S(Re²H)/S(Re¹⁸O); wherein the subscript “p” means that the data is obtained from water molecules in a sample of blood plasma and the subscript “e” means that the data is obtained from water molecules in a sample of erythrocyte.

Sₜₒₜₐₗ=Sₑ(rₑ)+Sₚ(rₚ).

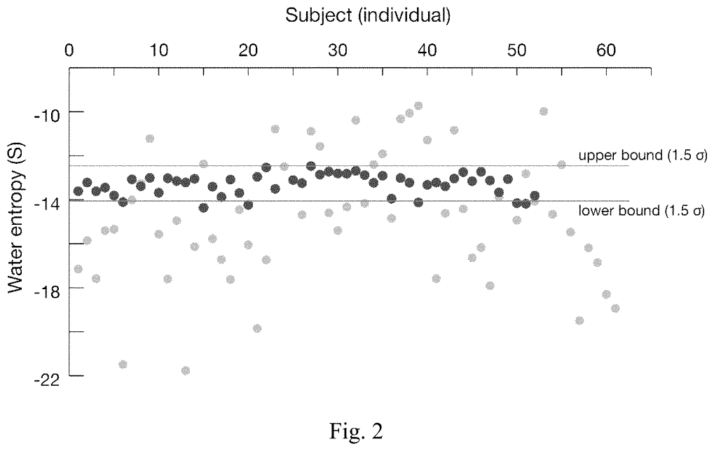

Then, plotting a diagram with the values of Sₜₒₜₐₗ at y-axis versus the number of subject at x-axis. The results are as shown in FIG. 2.

It can be clearly noted that the water entropy (Sₜₒₜₒₗ) derived from the subject of Group I (gray circle) is randomly distributed. However, the water entropy (Sₜₒₜₐₗ) derived from

Group II (dark circle) is distributed within a much narrower range, and a threshold can be defined.

Given this, the likelihood of suffering a renal disease of a subject suspected of the renal disease can be diagnosed by comparing the water entropy (Sₜₒₜₐₗ) with the threshold noted above. If the value falls within the range, then the subject may have potential of suffering from a renal disease.

### Example 2

Another model is applied in this example:

$$Body water index ( BWI ) = \frac{2 \times {δ}_{p}^{1 B} O + {δ}_{p}^{2} H}{2 \times {δ}_{e}^{1 B} O + {δ}_{e}^{2} H} .$$

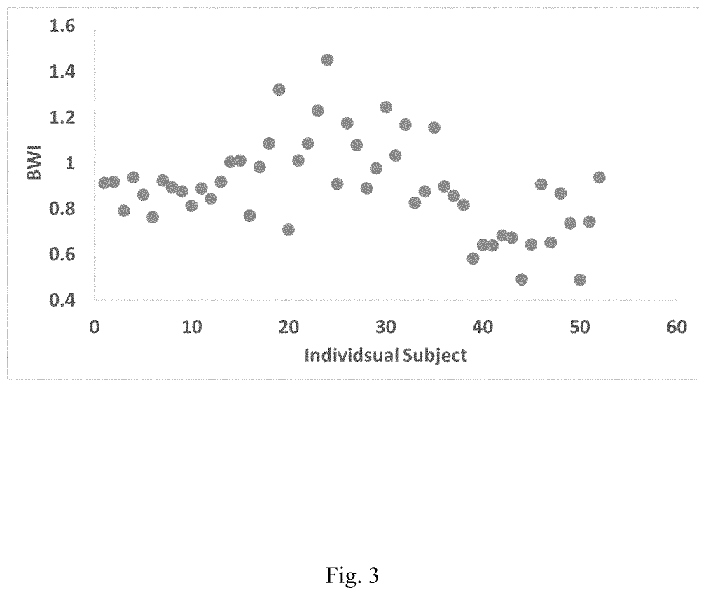

The results are as shown in FIG. 3.

### Example 3

Another model is applied in this example:

Body water index (BWI)=−3×δ¹⁸O+δ²H

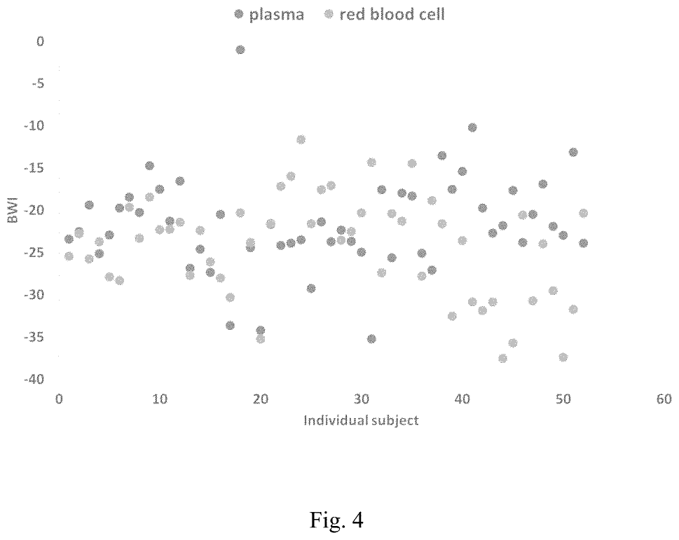

The results are as shown in FIG. 4.

### Example 4

Another model is applied in this example:

Body water index (BWI)=log(−δ¹⁸O) vs log(−δ² H)

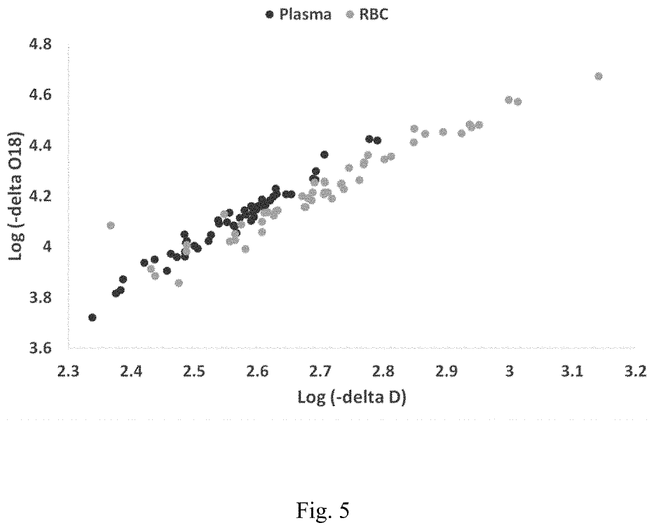

The results are as shown in FIG. 5.

### Example 5

Another model is applied in this example:

In[−(δₚ¹⁸O+δₑ¹⁸O)]vs In[−(δₚ²H+δₑ²H)]

Body water index (BWI)=

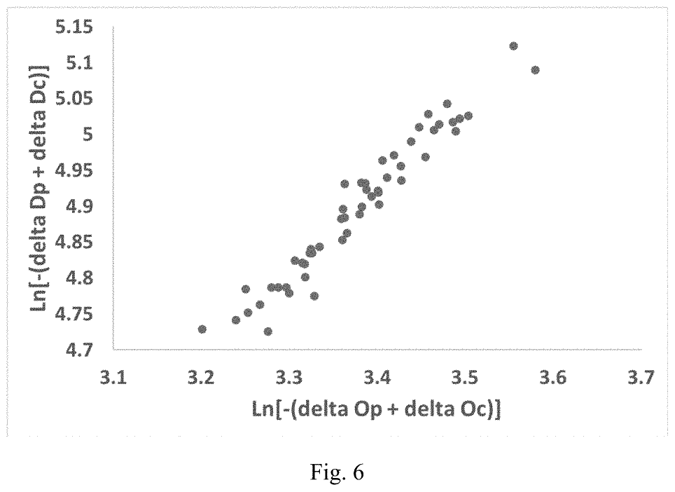

The results are as shown in FIG. 6.

### Example 6

Another model is applied in this example:

Body water index (BWI)=Rₚ¹⁸O×In(Rₚ¹⁸O)+Rₑ¹⁸O×In (Rₑ¹⁸O) vs Rₚ²H×In(Rₚ²H)+Rₑ²H×In(Rₑ²H)

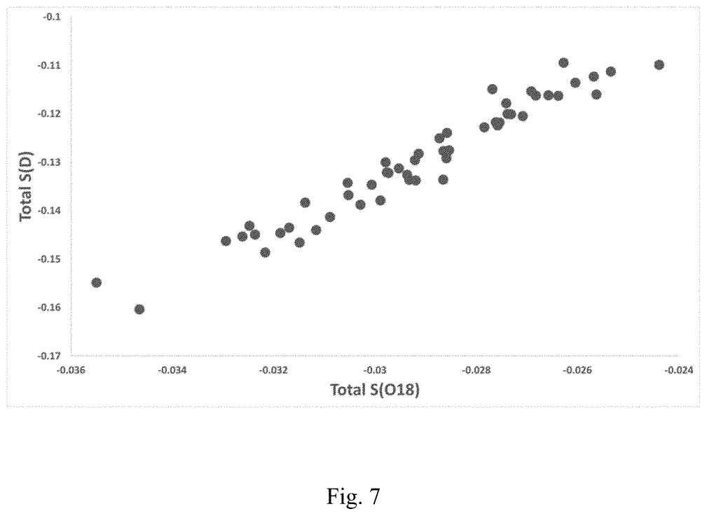

The results are as shown in FIG. 7.

### Example 7

Another model is applied in this example:

$$Body water index ( BWI ) = ( \frac{{R}_{p}^{2} H}{{R}_{p}^{18} O} ) \times Ln ( \frac{{R}_{p}^{2} H}{{R}_{p}^{18} O} ) + ( \frac{{R}_{e}^{2} H}{{R}_{e}^{18} O} ) \times Ln ( \frac{{R}_{e}^{2} H}{{R}_{e}^{18} O} )$$

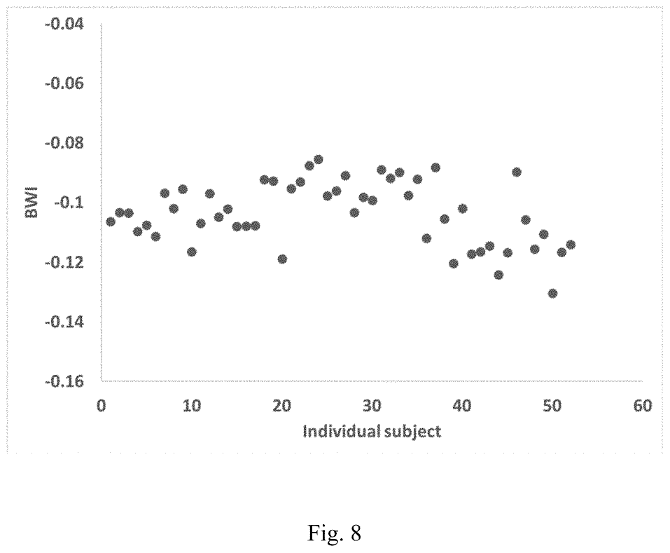

The results are as shown in FIG. 8.

A person of ordinary skill in the art of the subject disclosure should understand that variations and modification may be made to the teaching and the disclosure of the subject disclosure without departing from the spirit and scope of the subject application. Based on the contents above, the subject application intends to cover any variations and modification thereof with the proviso that the variations or modifications fall within the scope as defined in the appended claims or their equivalents.

What is claimed is:

1. A method for diagnosing renal disease, comprising:
receiving first data, second data and third data associated with water molecules;
selecting a first parameter associated with the first data and a second parameter associated with the second data;
establishing a relation between the first data and the second data based on the first parameter and the second parameter;
obtaining a threshold based on the relation between the first data and the second data; and
comparing a third parameter associated with the third data to the threshold to obtain a first probability of suffering a renal disease in a subject suspected of the renal disease; wherein:
the first data comprises values of δ¹⁸O and δ²H of water molecules in blood samples of subjects who are not suffered from renal disease;
the second data comprises values of δ¹⁸O and δ²H of water molecules in blood samples of subjects who are diagnosed of suffering renal disease and are subjected to treatment of hemodialysis;
the third data comprises values of δ¹⁸O and δ²H of water molecules in a blood sample of the subject suspected of the renal disease; and
the first, second and third parameters are derived from application of logarithm, addition, deduction, multiplication, division or any combinations thereof on the first, the second and the third data.

2. The method of claim 1, further comprising obtaining the first, the second and the third data from medical record, measurement of the blood samples, or both.

3. The method of claim 1, further comprising obtaining forms of entropy of the first and second parameters.

4. The method of claim 1, further comprising obtaining the threshold based on one or more statistic models.

5. The method of claim 4, wherein the statistic models comprise Student's t-test, F-test, Z-test, Analysis of variance (ANOVA) or any combinations thereof.

6. The method of claim 1, wherein establishing the relation between the first data and the second data comprises providing a two-dimensional plot with the first and second parameters as the axes.

7. The method of claim 6, further comprising expressing the threshold with standard deviation (σ) or variance (Υ²).

8. The method of claim 1, wherein the blood sample includes blood plasma, erythrocytes or both.

9. The method of claim 1, wherein the subjects associated with the second data have a family medical history of being subjected to treatment of hemodialysis, diabetes or both.

10. An apparatus, comprising:
a control unit; and
a memory including computer program code;
wherein the memory and the computer program code are configured to, with the control unit, cause the apparatus to perform:
receiving first data, second data and third data associated with water molecules;
selecting parameters associated with the first data and the second data;
establishing a relation between the first data and the second data based on a first parameter and a second parameter;
obtaining a threshold based on the relation between the first data and the second data; and
comparing a third parameter associated with the third data to the threshold to determine a first probability of suffering a renal disease in a subject suspected of the renal disease; wherein:
the first data comprises values of δ¹⁸O and δ²H of water molecules in blood samples of subjects who are not suffered from renal disease;
the second data comprises values of δ¹⁸O and δ²H of water molecules in blood samples of subjects who are diagnosed of suffering renal disease and are subjected to treatment of hemodialysis;
the third data comprises values of δ¹⁸O and δ²H of water molecules in a blood sample of the subject suspected of the renal disease; and
the parameters are derived from application of logarithm, addition, deduction, multiplication, division or any combinations thereof on the first, the second and the third data.

11. The apparatus of claim 10, further comprising expressing the parameters in the form of entropy.

12. The apparatus of claim 10, further comprising obtaining the threshold based on one or more statistic models.

13. The apparatus of claim 12, wherein the statistic models comprise Student's t-test, F-test, Z-test, Analysis of variance (ANOVA) and any combinations thereof.

14. The apparatus of claim 10, further comprising providing a two-dimensional plot with the first and second parameters as the axes to establish the relation between the first and the second data.

15. The apparatus of claim 14, further comprising expressing the threshold with standard deviation (σ) or variance (σ²).

16. The apparatus of claim 10, wherein the blood sample includes blood plasma, erythrocytes or both.

17. The apparatus of claim 10, wherein the subjects associated with the second data have a family medical history of being subjected to treatment of hemodialysis, diabetes or both.

18. The apparatus of claim 10, wherein the control unit comprises at least one receiving unit, at least one processing unit and at least one output unit.

19. The apparatus of claim 10, wherein the comparison of the third data to the threshold is conducted by a comparison unit.

20. A method for diagnosing renal disease using the apparatus of claim 10.
